(19) United States
(12) Patent Application Publication
(10) Pub. No.: US 2010/0227003 A1
(43) Pub. Date: Sep. 9, 2010

(54) Self-Preserving Compositions

(75) Inventors: Mandar V. Shah, Rockaway, NJ (US); Uday Doshi, Randolph, NJ (US); Ken T. Holeva, Phillipsburg, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: MCNEIL-PPC, INC., New Brunswick, NJ (US)

(21) Appl. No.: 12/784,746

(22) Filed: May 21, 2010

Related U.S. Application Data
(62) Division of application No. 11/475,617, filed on Jun. 27, 2006.

(51) Int. Cl. A61K 33/30; A61P 37/08

(52) U.S. Cl. 424/642; 424/641

(57) ABSTRACT

The invention provides self-preserving compositions and methods for their production.

### BACKGROUND OF THE INVENTION

1. Technical Field

The invention relates generally to ophthalmic, otic (for ears), and nasal compositions, and more particularly, to a self-preserving composition comprising an anti-microbial buffer and an anti-microbial metal ion. The invention further describes a general method to achieve self-preservation of other pharmaceutical products in which preservation is required or desirable. Examples of such products include, for example, intra-muscular injections, oral solutions, oral-care solutions, dental products, and the like.

2. Background Art

Ophthalmic, otic, and nasal compositions are employed for a wide range of indications, from the simple relief of dry or irritated eyes to the administration of therapeutic agents. Due to the multi-dose nature of these products, they are prone to contamination with microorganisms during their administration. Hence, these compositions carry with them a risk for introducing infectious agents to the eye, ear, or nasal passage. Such agents include bacteria and fungi, including yeasts. This is particularly true for ophthalmic products. However, the teachings of the present invention are applicable to other pharmaceutical or therapeutic dosage forms as well, particularly multi-dose products, such as multi-dose otic and nasal compositions.

The consequences of eye infections resulting from the use of an ophthalmic composition can be serious. In addition to unpleasant and uncomfortable symptoms such as redness, pain, excessive tearing and/or discharge, blurred vision, and increased light sensitivity, serious or untreated eye infections may result in permanent vision loss and/or the need for a corneal transplant.

Recent outbreaks of Fusarium (Fungal) keratitis, in which a majority of cases have been attributed to microbial growth in contact lens solutions, point out the need for ophthalmic solutions that do not promote the growth of microbial species. Optionally, such ophthalmic solutions would inhibit the growth of microbial species.

As noted above, multi-dose ophthalmic products are very likely to become contaminated during their administration. Hence, such products are required to be preserved. Commonly-used preservatives include, for example, benzalkonium chloride, (BAK); benzethonium chloride; benzyl alcohol; busan, cetrimide; chlorhexidine; chlorobutanol; edetate disodium; mercurial preservatives such as phenylmercuric nitrate, phenylmercuric acetate, and thimerosal; methylparabens and propylparabens; phenylethyl alcohol; Purite® (stabilized oxychloro compound); sodium perborate; sorbic acid and potassium sorbate; polyaminopropyl buguanide; polyquaternium-1; polyhexamethylene biguanide (PHMB); and polyvinylpyrrolidone (PVP)-iodine complexes.

The most commonly employed ophthalmic preservative is BAK. However, long-term and/or frequent use of BAK-preserved products have been associated with ocular toxicity, such as damage to epithelial surface and decreased tolerability due to irritation (Berdy et al., 1992; Noecker 2001).

To reduce the toxicity of BAK, gentler, milder, or disappearing preservatives have been developed. An example of a gentler preservative is Polyquad®, available from Alcon (see U.S. Pat. No. 4,525,346). Examples of disappearing preservatives include stabilized hydrogen peroxide available from Ciba Vision, which disappears upon administration into the eye (see U.S. Pat. No. 5,725,887) and the stabilized oxy-chloro complex (Purite®) available from Allergan (see U.S. Patent Application Publication No. 2004/0137079), which also breaks down into harmless products upon application to the eye. Further, Alcon utilizes a borate-polyol complex to increase the preservative efficacy of its formulations (see U.S. Pat. No. 5,342,620), especially the ones with Polyquad®.

Despite these advances, to the knowledge of Applicants, self-preserved compositions (i.e., compositions free or substantially free of preservatives) have not been developed. Accordingly, there is a need for a self-preserving composition, optionally one comprising ingredients commonly used in ophthalmic, otic, and/or nasal preparations. Also or optionally, such a composition would have a pH at or around the physiologic pH of 7.4.

### SUMMARY OF THE INVENTION

The invention provides self-preserved compositions and methods for their production. In particular, compositions according to the invention combine several physical and/or chemical parameters to create a vehicle that is hostile to microorganisms but innocuous to the eye, ear, nose, or other site of application. More particularly, unique combinations of commonly known parameters listed below are described, which yield a self-preserved composition. These parameters include:

Antimicrobial buffer (e.g., borate);

Antimicrobial ion (e.g., zinc);

pH and tonicity (osmolality);

surfactant (e.g., polyoxyethylene sorbitan monooleate);

antioxidant (e.g., ascorbic acid);

chelating agent (e.g. ethylenediaminetetracetic acid (EDTA));

absence of certain ions (e.g., magnesium, calcium, etc.).

It should be noted that to achieve self-preservation, it may not be possible or beneficial to manipulate or utilize all of the above parameters in a single composition. However, it may be necessary to manipulate or utilize more than one of these parameters in order to achieve self-preservation. Details of the manipulation and/or utilization of these parameters, their impact (directional and quantitative), and the resolution of their potential incompatibilities are described below.

A first aspect of the invention provides a self-preserving composition comprising: an anti-microbial buffer; and an anti-microbial metal ion, wherein a pH of the composition is between about 6.0 and about 8.0 and an osmolality of the composition is between about 200 and about 400 mOsm/kg.

A second aspect of the invention provides a method for preserving a composition comprising: incorporating into the composition an antimicrobial buffer; and incorporating into the composition an antimicrobial metal ion.

A third aspect of the invention provides a composition comprising: an antimicrobial buffer; ascorbic acid; a source of zinc ions; and polyoxyethylene sorbitan monooleate, wherein precipitation of zinc is inhibited by the ascorbic acid.

A fourth aspect of the invention provides a method for preserving a composition, the method comprising removing from the composition at least one species of ion beneficial to the growth of a microorganism, wherein the species of ion is selected from a group consisting of: potassium ions, calcium ions, and magnesium ions.

A fifth aspect of the invention provides a method for treating an ocular allergy symptom in an individual, the method comprising: administering to a surface of an eye of an individual a composition comprising an effective amount of zinc, wherein the zinc is capable of precipitating from the surface of the eye at least one protein causing a symptom of an ocular allergic reaction.

A sixth aspect of the invention provides an ophthalmic composition comprising: a source of zinc, wherein the source of zinc is capable of precipitating from a surface of an eye at least one protein capable of causing at least one symptom of an ocular allergic reaction.

The illustrative aspects of the present invention are designed to solve the problems herein described and other problems not discussed, which are discoverable by a skilled artisan.

### BRIEF DESCRIPTION OF THE DRAWINGS

These and other features of this invention will be more readily understood from the following detailed description of the various aspects of the invention taken in conjunction with the accompanying drawings that depict various embodiments of the invention, in which:

FIG. 1 shows a flow diagram of an illustrative method for preparing a composition according to the invention.

FIG. 2 shows a flow diagram of an illustrative method for preparing a composition containing a lipophilic compound according to the invention.

It is noted that the drawings of the invention are not to scale. The drawings are intended to depict only typical aspects of the invention, and therefore should not be considered as limiting the scope of the invention. In the drawings, like numbering represents like elements between the drawings.

### DETAILED DESCRIPTION

The self-preserving compositions of the present invention can comprise, consist of, or consist essentially of the essential elements and limitations of the invention described herein, as well any of the additional or optional ingredients, components, or limitations described herein.

All percentages, parts and ratios are based upon the total weight of the self-preserving composition of the present invention, unless otherwise specified.

As used herein, the term “borate” includes boric acid, its salts, other pharmaceutically-acceptable borates, their salts, and combinations thereof. These include, for example, boric acid, sodium borate, potassium borate, calcium borate, magnesium borate, manganese borate, and other such borate salts.

As used herein, the phrase “substantially free of preservatives,” as applied to compositions of the invention, shall include compositions which include one or more preservative, each of which being present in a concentration insufficient to achieve a preservative effect, as defined by the United States Pharmacopeia (USP) and as shown in Table 1, which provides required reductions in counts of index bacteria and fungi species using the USP Preservative Efficacy Test (PET).

TABLE 1
USP Requirements for PET

| | Log Reduction | | |
|---|---|---|---|
| | 7 Days | 14 Days | 28 Days |
| | Incubation | Incubation | Incubation |
| Bacteria | | | |
| E. coli | 1 | 3 | No Increase |
| S. aureus | 1 | 3 | No Increase |
| Ps. auruginosa | 1 | 3 | No Increase |
| Fungi | | | |
| C. albicans | No Increase | No Increase | No Increase |
| A. niger | No Increase | No Increase | No Increase |

Alternatively, “substantially free of preservatives,” as applied to compositions of the invention, shall include compositions which include one or more preservatives in Table 2 below, but which are present in a concentration less than the range shown.

TABLE 2
Commonly-Used Preservatives and Their Typical Ranges

| COMMONLY-USED PRESERVATIVES | | TYPICAL % RANGE |
|---|---|---|
| 1. | Benzalkonium Chloride (BAK) | 0.004-0.02% |
| 2. | Banzethonium Chloride | 0.01-0.02% |
| 3. | Benzyl Alcohol | 0.1% |
| 4. | Busan | 0.001-0.006% |
| 5. | Cetrimide | 0.005% |
| 6. | Chlorhexidine | 0.005-0.1% |
| 7. | Chlorobutanol | 0.15%-0.55% |
| 8. | Edetate Disodium | 0.01-0.25% |
| 9. | Mercurial Preservatives | |
| | Phenylmercuric Nitrate | 0.002-0.004% |
| | Phenylmercuric Acetate | 0.0008% |
| | Thimerosal | 0.001-0.2% |
| 10. | Methylparabens and Propylparabens | Methylparabens - 0.03-1% |
| | | Propylparabens - up to 0.01% |
| 11. | Phenylethyl Alcohol | 0.25-0.5% |
| 12. | Purite ® (Stabilized Oxychloro | 0.005% |
| | Compound) | |
| 13. | Sorbic Acid/Potassium Sorbate | 0.1-0.25% |
| 14. | Polyaminopropyl Biguanide | 0.00005-0.0015% |
| 15. | Polyquatemium-1 | 0.001% |
| 16. | Polyhexamethylene biguanide | 0.02-0.05% |
| | (PHMB) | |
| 17. | PVP-Iodine complex | 0.0005-0.001% |

Optionally, each such preservative is present in a concentration less than 75% of such a concentration, optionally less than about 50% of such a concentration, and optionally less than about 25% of such a concentration. For example, in known ophthalmic compositions, benzalkonium chloride (BAK) is typically present in a concentration between about 0.004% and about 0.02%. Thus, a self-preserved ophthalmic composition according to the invention may further comprise a quantity of BAK at a concentration less than between about 0.004% and about 0.02%, optionally between about 0.003% and about 0.015%, optionally between about 0.002% and about 0.01%, and optionally between about 0.001% and about 0.005%.

However, it should be understood that the inclusion of a preservative, such as those shown in Table 2, in a composition of the invention shall not be necessary in order to preserve the composition. Specifically, the inclusion of such a preservative in a composition of the invention shall not be necessary, and alone such a preservative shall be insufficient, to achieve USP standards regarding preservation.

A number of PETs were performed to investigate the antimicrobial effect of various combinations of antimicrobial buffer, pH, and osmolality (tonicity). Table 3 shows the compositions of various antimicrobial compositions, while Table 4 shows the effect of each composition on each of the index species. As described herein, compositions according to the invention include borate buffers as a non-preservative buffer. The phrase “non-preservative buffer” as used herein means a buffer which at its buffering concentration fails to achieve a preservative effect, as defined by the United States Pharmacopeia (USP) and as shown in Table 1. Other non-preservative buffer systems having antimicrobial properties may similarly be used, such as, an ethanolamine/biguanide buffer, a tricine buffer, a cetylpyridinium chloride buffer, or a cationic polysaccharide buffer.

TABLE 3
Compositions of Antimicrobial Compositions

| | Amount (% w/w) | | | |
|---|---|---|---|---|
| | pH 6.5 | pH 7.5 | pH 6.5 | pH 7.5 |
| | & 225 | & 225 | & 290 | & 290 |
| Ingredients | mOsm/Kg | mOsm/Kg | mOsm/Kg | mOsm/Kg |
| Boric Acid | 0.96 | 0.80 | 0.96 | 0.80 |
| Sodium Borate | 6.04 | 0.20 | 0.04 | 0.20 |
| Sodium chloride | 0.20 | 0.24 | 0.40 | 0.46 |
| Purified Water | q.s. to 100 | q.s. to 100 | q.s. to 100 | q.s. to 100 |

TABLE 4
PET results for Antimicrobial Compositions

| No. of | | | | | |
|---|---|---|---|---|---|
| Days | | Log Reduction | | | |
| incubated | Organism | pH 6.5 & 225 mOsm/Kg | pH 7.5 & 225 mOsm/Kg | pH 6.5 & 290 mOsm/Kg | pH 7.5 & 290 mOsm/Kg |
| 1 Day | E. coli | 1.1 | 1.8 | 0.1 | 0.2 |
| | S. aureus | 0.4 | 2.1 | 0.2 | 0.8 |
| | Ps. aerug. | 2.1 | 5.3 | 1.0 | 4.4 |
| | C. albicans | 0.1 | 0.0 | 0.2 | 0.1 |
| | A. niger | 0.4 | 0.5 | 0.5 | 0.5 |
| 3 days | E. coli | 1.0 | 4.0 | 1.1 | 2.8 |
| | S. aureus | 1.4 | 4.2 | 1.2 | 2.0 |
| | Ps. aerug. | 3.2 | 5.3 | 1.4 | 5.3 |
| | C. albicans | 0.2 | 0.2 | 0.2 | 0.4 |
| | A. niger | 0.5 | 1.7 | 1.5 | 1.9 |
| 7 days | E. coli | 2.5 | 5.3 | 1.8 | 5.3 |
| | S. aureus | 5.4 | 5.8 | 4.5 | 4.4 |
| | Ps. aerug. | 5.3 | 5.3 | 2.0 | 5.3 |
| | C. albicans | 0.4 | 1.1 | 0.4 | 1.2 |
| | A. niger | 0.2 | 1.7 | 0.9 | 0.2 |
| 14 days | E. coli | 5.3 | 5.3 | 5.3 | 5.3 |
| | S. aureus | 5.4 | 5.8 | 5.4 | 5.4 |
| | Ps. aerug. | 5.3 | 5.3 | 3.3 | 5.3 |
| | C. albicans | 1.6 | 4.1 | 1.7 | 3.3 |
| | A. niger | 0.2 | 1.7 | 0.9 | 0.3 |
| 21 days | E. coli | 5.3 | 5.3 | 5.3 | 5.3 |
| | S. aureus | 5.4 | 5.8 | 5.4 | 5.4 |
| | Ps. aerug. | 5.3 | 5.3 | 4.6 | 5.3 |
| | C. albicans | 3.7 | 5.4 | 3.7 | 5.4 |
| | A. niger | 0.2 | 0.9 | 1.1 | 0.2 |
| 28 days | E. coli | 5.3 | 5.3 | 5.3 | 5.3 |
| | S. aureus | 5.4 | 5.8 | 5.4 | 5.4 |
| | Ps. aerug. | 5.3 | 5.3 | 5.3 | 5.3 |
| | C. albicans | 5.4 | 5.4 | 5.4 | 5.4 |
| | A. niger | 1.2 | 0.8 | 0.9 | 0.3 |

As can be seen in Table 4, an osmolality of 225 mOsm/kg improves the reduction in counts of E. coli and Ps. Aerug., as compared to an osmolality of 290 mOsm/kg. This may be attributed to the fact that bacteria have osmolalities of about 290 mOsm/kg. As such, bacteria become weakened as osmolality decreases and become more susceptible to the antimicrobial agents. These differences in antimicrobial effectiveness are better observed during earlier periods of incubation, e.g., 1-3 days. Greater preservative efficacy is observed at pH of 7.5 than at pH 6.5.

Table 5 shows the effect of the inclusion of various surfactants (cremophor EL, polysorbate 80, and pluronic F108) on the PET of an aqueous composition comprising 0.96% boric acid, 0.04% sodium borate, 0.1% EDTA, and 0.5% glycerin.

TABLE 5
Effect of Surfactants on PET

| | | Log Reduction | | | |
|---|---|---|---|---|---|
| | | | | Composition | |
| No. of | | | Composition | with 1% | Composition |
| Days | | Composition | with 1% | polysorbate | with pluronic |
| incubated | Organism | with no Surfactant | cremophor | 80 | F108 |
| 7 Day | E. coli | 0.79 | 1.04 | 1.93 | 1.12 |
| | S. aureus | 0.64 | 0.64 | 1.58 | No increase |
| | Ps. aerug. | 3.32 | 3.54 | 3.89 | 3.50 |
| | C. albicans | No decrease | 0.02 | 0.07 | 0.00 |
| | A. niger | 1.23 | 1.10 | 0.58 | No increase |
| 14 days | E. coli | 2.08 | 1.94 | 3.42 | 2.71 |
| | S. aureus | 1.28 | 1.35 | 2.91 | 0.99 |
| | Ps. aerug. | 4.94 | 4.94 | 4.94 | 4.94 |
| | C. albicans | 0.88 | 1.10 | 2.67 | 1.80 |
| | A. niger | 0.95 | 1.20 | 0.80 | 0.84 |
| 21 days | E. coli | 3.30 | 1.95 | 5.04 | 3.23 |
| | S. aureus | 1.54 | 1.96 | 4.98 | 1.28 |
| | Ps. aerug. | 4.94 | 4.94 | 4.94 | 4.94 |
| | C. albicans | 1.77 | 2.11 | 5.04 | 3.38 |
| | A. niger | 0.58 | 1.10 | 0.44 | 0.44 |
| 28 days | E. coli | 3.4 | 3.1 | 5.0 | 4.9 |
| | S. aureus | 2.8 | 3.4 | 5.0 | 2.2 |
| | Ps. aerug. | 4.9 | 4.9 | 4.9 | 4.9 |
| | C. albicans | 4.2 | 4.9 | 5.0 | 4.6 |
| | A. niger | 0.8 | 0.7 | 0.6 | 0.4 |

As can be seen in Table 5, compositions including cremophor EL or pluronic F108 exhibited no or modest decreases in counts of index species, as compared to the composition having no surfactant. The inclusion of polysorbate 80 (polyoxyethylene sorbitan monooleate), however, results in a significant reduction counts of most index species in all time periods. One exception is the effect on A. Niger, which was less than that of the composition having no surfactant.

In order to assess the affect of chelating agents on PET, EDTA was added to the 1% polysorbate 80 composition of Table 5. The results are shown in Table 6.

TABLE 6
Effect of Chelating Agent on PET

Log Reduction

| No. of Days | | Composition | Composition |
|---|---|---|---|
| incubated | Organism | with EDTA | without EDTA |
| 7 Day | E. coli | 1.93 | 0.4 |
| | S. aureus | 1.58 | 3.8 |
| | Ps. aerug. | 3.89 | 5.1 |
| | C. albicans | 0.07 | 0.4 |
| | A. niger | 0.58 | 1.1 |
| 14 days | E. coli | 3.42 | 2.2 |
| | S. aureus | 2.91 | 5.4 |
| | Ps. aerug. | 4.94 | 5.1 |
| | C albicans | 2.67 | 1.7 |
| | A. niger | 0.80 | 1.0 |
| 21 days | E. coli | 5.04 | 5.4 |
| | S. aureus | 4.98 | 5.4 |
| | Ps. aerug. | 4.94 | 5.1 |
| | C. albicans | 5.04 | 5.5 |
| | A. niger | 0.44 | 1.2 |
| 28 days | E. coli | 5.0 | 5.4 |
| | S. aureus | 5.0 | 5.4 |
| | Ps. aerug. | 4.9 | 5.1 |
| | C. albicans | 5.0 | 5.5 |
| | A. niger | 0.6 | 1.6 |

As can be seen from Table 6, the effect of EDTA on PET is complex. The addition of EDTA resulted in a significant reduction in the counts of E. coli, as compared to the composition without EDTA. However, the presence of EDTA yielded a reduction in PE for S. Aureus and Ps. Aerug.

The additional effect of antioxidants on PET is shown in Table 7, wherein the composition including EDTA in Table 6 was tested against a similar composition further including 0.01% ascorbic acid.

TABLE 7
Antioxidant Effect on PET

Log Reduction

| | | Composition | Composition |
|---|---|---|---|
| No. of Days | | without | with |
| incubated | Organism | Ascorbic Acid | Ascorbic Acid |
| 7 Day | E. coli | 1.93 | 2.7 |
| | S. aureus | 1.58 | 1.7 |
| | Ps. aerug. | 3.89 | 5.1 |
| | C. albicans | 0.07 | 1.3 |
| | A. niger | 0.58 | 1.2 |
| 14 days | E. coli | 3.42 | 5.4 |
| | S. aureus | 2.91 | 5.4 |
| | Ps. aerug. | 4.94 | 5.1 |
| | C. albicans | 2.67 | 5.5 |
| | A. niger | 0.80 | 1.2 |
| 21 days | E. coli | 5.04 | 5.4 |
| | S. aureus | 4.98 | 5.4 |
| | Ps. aerug. | 4.94 | 5.1 |
| | C. albicans | 5.04 | 5.5 |
| | A. niger | 0.44 | 1.2 |
| 28 days | E. coli | 5.0 | 5.4 |
| | S. aureus | 5.0 | 5.4 |
| | Ps. aerug. | 4.9 | 5.1 |
| | C. albicans | 5.0 | 5.5 |
| | A. niger | 0.6 | 1.5 |

As can be seen, the presence of ascorbic acid results in a significant improvement in PET for all index species. The effect on E. coli and S. aureus during early periods (7 days and 14 days) was particularly significant. These results are attributable, at least in part, to the removal of oxygen from the composition by ascorbic acid, making it difficult for aerobic organisms to grow. While ascorbic acid was employed in this study, any antioxidant capable of reducing and/or removing dissolved oxygen from the composition would exhibit similar results.

Examples of suitable antioxidants include, but are not limited to, ascorbic acid, sodium bisulfite, sodium metabisulfite, other potassium and sodium salts of sulfurous acid, thiourea, isoascorbic acid, thioglycerol, and cysteine hydrochloride. bht (butylated hydroxytoluene), bha (butylated hydroxyanisole), tocopherals alkyl gallates and nordihydroguaiaretic acid, synergistic agents such as citric acid, ethylenediaminetetraacetic acid salts, lecithin, phosphoric acid, tartaric acid, thiodipropionic acid, and mixtures thereof.

In certain embodiments of the present invention, the antioxidant is selected from the group consisting of ascorbic acid, sodium bisulfite, sodium metabisulfite, other potassium and sodium salts of sulfurous acid, thiourea and mixtures thereof.

To assess the effect of antimicrobial metal ions on PE, two antimicrobial compositions were tested, one containing an antimicrobial ion and one not containing an antimicrobial ion. The formulations of the two compositions are shown in Table 8. In order to avoid complexing of zinc by EDTA, the composition containing zinc did not contain EDTA. The respective results of each composition on PET are shown in Table 9. It should be noted that while the results below are shown for zinc, other metal ions exhibiting antimicrobial properties would yield similar results. Such metal ions include, for example, a silver ion, a nickel ion, an iron ion, a cobalt ion, a copper ion, a manganese ion, a gold ion, a chromium ion, a platinum ion, a palladium ion or mixtures thereof.

TABLE 8
Compositions with and without Antimicrobial Ion

Amount (% w/w)

| | Composition | Composition |
|---|---|---|
| Ingredients | without zinc | with zinc |
| Boric Acid | 0.96 | 0.96 |
| Sodium Borate | 0.04 | 0.04 |
| EDTA | 0.1 | — |
| Ascorbic Acid | 0.01 | 0.01 |
| Zinc chloride | — | 0.01 |
| polysorbate 80 | 1.0 | 1.0 |
| Glycerin | 0.5 | 0.5 |
| Purified Water | q.s. to 100 | q.s. to 100 |

TABLE 9
Antimicrobial Ion Effect on PET

Log Reduction

| No. of Days | | Composition | Composition |
|---|---|---|---|
| incubated | Organism | without zinc | with zinc |
| 7 Day | E. coli | 2.7 | 5.4 |
| | S. aureus | 1.7 | 5.4 |
| | Ps. aerug. | 5.1 | 5.1 |
| | C. albicans | 1.3 | 1.3 |
| | A. niger | 1.2 | 1.2 |
| 14 days | E. coli | 5.4 | 5.4 |
| | S. aureus | 5.4 | 5.4 |
| | Ps. aerug. | 5.1 | 5.1 |
| | C. albicans | 5.5 | 5.5 |
| | A. niger | 1.2 | 1.0 |
| 21 days | E. coli | 5.4 | 5.4 |
| | S. aureus | 5.4 | 5.4 |
| | Ps. aerug. | 5.1 | 5.1 |
| | C. albicans | 5.5 | 5.5 |
| | A. niger | 1.2 | 0.9 |
| 28 days | E. coli | 5.4 | 5.4 |
| | S. aureus | 5.4 | 5.4 |
| | Ps. aerug. | 5.1 | 5.1 |
| | C. albicans | 5.5 | 5.5 |
| | A. niger | 1.5 | 1.1 |

As can be seen in Table 9, the effect of antimicrobial zinc ions on PET was greatest for E. coli and S. aureus, where maximum PET results were achieved by day 7. As compared to the composition containing ascorbic acid in Table 7, maximum PET results were achieved 7 days earlier using the composition containing zinc.

As noted above, others have developed ophthalmic compositions comprising borate-polyol complexes. In order to assess the PET effect of such complexes on compositions of the present invention, a number of compositions, shown in Table 10, were tested. PET results are shown in Table 11.

TABLE 10
Compositions with and without Borate-Polyol Complexes

| | Amount (% w/w) | | | |
|---|---|---|---|---|
| | Control | Solution | Solution | Solution |
| | solution | with AA | with AA | with AA |
| | with AA | and Zn but | and Zn but | and Zn but |
| Ingredients | and Zn | w/o poly | w/o gly | w/o poly & gly |
| Boric Acid | 0.96 | 0.96 | 0.96 | 0.96 |
| Sodium Borate | 0.04 | 0.04 | 0.04 | 0.04 |
| Zinc chloride | 0.01 | 0.01 | 0.01 | 0.01 |
| Sodium Chloride | — | — | 0.17 | 0.17 |
| Glycerin | 0.05 | 0.05 | — | — |
| Ascorbic Acid | 0.01 | 0.01 | 0.01 | 0.01 |
| Polysorbate 80 | 1.0 | — | 1.0 | — |
| Purified Water | q.s. to 100 | q.s. to 100 | q.s. to 100 | q.s. to 100 |

TABLE 11
Borate-Polyol Complex Effect on PET

| | | Log Reduction | | | |
|---|---|---|---|---|---|
| No. of | | Control | Solution with | Solution with | Solution with |
| Days | | solution with | AA and Zn | AA and Zn | AA and Zn but |
| incubated | Organism | AA and Zn | but w/o poly | but w/o gly | w/o poly & gly |
| 7 days | E. coli | 5.5 | 5.5 | 5.5 | 5.5 |
| | S. aureus | 5.4 | 5.4 | 5.4 | 4.7 |
| | Ps. aerug. | 5.3 | 5.3 | 5.3 | 5.3 |
| | C. albicans | 0.7 | 0.4 | 0.3 | 0.3 |
| | A. niger | 1.3 | 1.0 | 0.9 | 1.0 |
| 14 days | E. coli | 5.5 | 5.5 | 5.5 | 5.5 |
| | S. aureus | 5.4 | 5.4 | 5.4 | 5.4 |
| | Ps. aerug. | 5.3 | 5.3 | 5.3 | 5.3 |
| | C. albicans | 2.5 | 1.8 | 2.7 | 3.5 |
| | A. niger | 1.4 | 1.4 | 1.1 | 0.9 |
| 21 days | E. coli | 5.5 | 5.5 | 5.5 | 5.5 |
| | S. aureus | 5.4 | 5.4 | 5.4 | 5.4 |
| | Ps. aerug. | 5.3 | 5.3 | 5.3 | 5.3 |
| | C. albicans | 5.4 | 4.2 | 5.4 | 5.4 |
| | A. niger | 2.6 | 2.5 | 1.4 | 1.4 |
| 28 days | E. coli | 5.5 | 5.5 | 5.5 | 5.5 |
| | S. aureus | 5.4 | 5.4 | 5.4 | 5.4 |
| | Ps. aerug. | 5.3 | 5.3 | 5.3 | 5.3 |
| | C. albicans | 5.4 | 5.4 | 5.4 | 5.4 |
| | A. niger | 3.4 | 2.7 | 1.1 | 1.3 |

As can be seen in Table 11, the presence of a borate-polyol complex has little or no effect on PET in a composition already comprising an antioxidant (ascorbic acid) and antimicrobial metal ion (zinc). In addition, the presence or absence of such borate-polyol complexes has no effect on the ability of a composition to meet USP requirements shown in Table 1.

Often, ophthalmic compositions will include a demulcent for relieving irritation and/or inflammation. Suitable demulcents include, but are not limited to, cellulose derivatives such as carboxymethylcellulose sodium, hydroxyethyl cellulose, hydroxypropyl methylcellulose, methylcellulose; dextran 70; gelatin; polyols such as glycerin, polyethylene glycol 300, polyethylene glycol 400, polysorbate 80, propylene glycol; polyvinyl alcohol; Hyaluronic acid; and povidone (polyvinyl pyrrolidone). Mixtures of the above listed demulcents can also be used. “Optionally, viscosity modifying agents may also be included with the above mentioned demulcents. These viscosity modifiers include, but are not limited to, polymers such as biopolymers, such as chondoritin sulfate and chitosan; synthestic polymers such as polyacrylic acid; gums such as xanthan gum and guar gum; and tamarind seed polymer.” Table 12 shows formulations of two demulcent-containing compositions, one containing hydroxymethyl propylcellulose (HPMC) and the other hyaluronic acid (HA), and a vehicle including ascorbic acid and zinc chloride. Table 13 shows the effect of each demulcent on PET.

TABLE 12
Demulcent-Containing Compositions

| | Amount (% w/w) | | |
|---|---|---|---|
| | AA, Zn based | HPMC containing | HA containing |
| Ingredients | vehicle | Product | Product |
| HPMC | — | 0.36 | — |
| PEG | — | 1.0 | — |
| Glycerin | — | 0.2 | — |
| Hyaluronic Acid | — | — | 0.2 |
| Boric Acid | 0.96 | 0.96 | 0.96 |
| Sodium Borate | 0.04 | 0.04 | 0.04 |
| Ascorbic Acid | 0.01 | 0.01 | 0.01 |
| Zinc chloride | 0.01 | 0.01 | 0.01 |
| Sodium Chloride | 0.18 | — | 0.18 |
| Purified Water | q.s. to 100 | q.s. to 100 | q.s. to 100 |

TABLE 13
Demulcent Effect on PET

| | | Log Reduction | | |
|---|---|---|---|---|
| | | | HPMC | HA |
| No. of Days | | AA, Zn based | containing | containing |
| incubated | Organism | vehicle | Product | Product |
| 24 hrs | E. coli | 3.9 | 1.0 | 3.6 |
| | S. aureus | 0.0 | 1.0 | 1.6 |
| | Ps. aerug. | 0.1 | 2.1 | 3.8 |
| | C. albicans | 0.2 | 0.0 | −0.1 |
| | A. niger | 1.3 | 1.0 | 1.3 |
| 7 Day | E. coli | 5.4 | 5.4 | 5.4 |
| | S. aureus | 5.0 | 5.4 | 5.4 |
| | Ps. aerug. | 5.3 | 5.3 | 5.3 |
| | C. albicans | 1.2 | 1.2 | 1.3 |
| | A. niger | 1.2 | 1.7 | 1.4 |
| 14 days | E. coli | 5.4 | 5.4 | 5.4 |
| | S. aureus | 5.4 | 5.4 | 5.4 |
| | Ps. aerug. | 5.3 | 5.3 | 5.3 |
| | C. albicans | 4.8 | 2.2 | 3.7 |
| | A. niger | 1.4 | 1.8 | 1.6 |
| 21 days | E. coli | 5.4 | 5.4 | 5.4 |
| | S. aureus | 5.4 | 5.4 | 5.4 |
| | Ps. aerug. | 5.3 | 5.3 | 5.3 |
| | C. albicans | 5.5 | 4.9 | 5.5 |
| | A. niger | 1.1 | 4.0 | 1.6 |
| 28 days | E. coli | 5.4 | 5.4 | 5.4 |
| | S. aureus | 5.4 | 5.4 | 5.4 |
| | Ps. aerug. | 5.3 | 5.3 | 5.3 |
| | C. albicans | 5.5 | 5.1 | 5.5 |
| | A. niger | 1.1 | 2.5 | 1.4 |

During the first 24 hours, the effect of HPMC on PET is mixed. PET improved in two species and worsened in three species. During the same period, HA improved PET in two species, worsened PET in two species, and had no effect in another. During later periods, both HPMC and HA improved PET in A. niger and worsened PET in C. albicans. In other species, neither demulcent affected PET beyond the 7 day period.

Because HPMC is available in both high viscosity and low viscosity varieties, it was unclear whether the viscosity of the HPMC used would affect PET. The effect of both polyethylene glycol (PEG) and glycerin on high- and low-viscosity HPMC compositions was concurrently tested. The formulation of each composition is shown in Table 14 and its effect on PET in Table 15.

TABLE 14
High- and Low-Viscosity HPMC Compositions
with and without PEG and Glycerin

| | Amount (% w/w) | | | |
|---|---|---|---|---|
| | High | High | Low | Low |
| | Viscosity | Viscosity | Viscosity | Viscosity |
| | HPMC with | HPMC w/out | HPMC with | HPMC w/out |
| Ingredients | PEG & GLY | PEG & GLY | PEG & GLY | PEG & GLY |
| Hypermelose | 0.36 | 0.36 | 0.36 | 0.36 |
| (E4M) | | | | |
| Boric Acid | 0.75 | 0.75 | 0.75 | 0.75 |
| Sodium Borate | 0.21 | 0.21 | 0.21 | 0.21 |
| Zinc Chloride | 0.01 | 0.01 | 0.01 | 0.01 |
| Ascorbic Acid | 0.1 | 0.1 | 0.1 | 0.1 |
| Glycerin | 0.25 | — | 0.25 | — |
| Polyethylene | 1.15 | — | 1.15 | — |
| Glycol 400 | | | | |
| Potassium | 0.025 | 0.025 | 0.025 | 0.025 |
| Chloride | | | | |
| Magnesium | 0.001 | 0.001 | 0.001 | 0.001 |
| Chloride | | | | |
| Sodium | 0.001 | 0.001 | 0.001 | 0.001 |
| chloride | | | | |
| Dextrose | 0.001 | 0.001 | 0.001 | 0.001 |
| Sodium Lactate | 0.005 | 0.005 | 0.005 | 0.005 |
| 60% solution | | | | |
| Glycine | 0.00002 | 0.00002 | 0.00002 | 0.00002 |
| Purified water | q.s to 100 | q.s to 100 | q.s to 100 | q.s to 100 |

TABLE 15
Effect of High- and Low-Viscosity HPMC, PEG, and Glycerin (GLY) on PET

| | | Log Reduction | | | |
|---|---|---|---|---|---|
| | | | | Low | Low |
| No. of | | High Viscosity | High Viscosity | Viscosity | Viscosity |
| Days | | HPMC with | HPMC w/out | HPMC with | HPMC w/out |
| incubated | Organism | PEG & GLY | PEG & GLY | PEG & GLY | PEG & GLY |
| 7 days | E. coli | 2.8 | 5.3 | 5.3 | 5.3 |
| | S. aureus | 4.7 | 3.7 | 5.1 | 5.1 |
| | Ps. aerug. | 5.2 | 3.2 | 4.9 | 3.4 |
| | C. albicans | 0.1 | 0.0 | 0.1 | 0.1 |
| | A. niger | 0.3 | 0.4 | 0.5 | 0.6 |
| 14 days | E. coli | 5.3 | 5.3 | 5.3 | 5.3 |
| | S. aureus | 5.1 | 5.1 | 5.1 | 5.1 |
| | Ps. aerug. | 5.3 | 5.3 | 5.3 | 5.3 |
| | C. albicans | 1.9 | 1.2 | 1.0 | 1.9 |
| | A. niger | 1.1 | 2.8 | 1.5 | 0.9 |
| 21 days | E. coli | 5.3 | 5.3 | 5.3 | 5.3 |
| | S. aureus | 5.1 | 5.1 | 5.1 | 5.1 |
| | Ps. aerug. | 5.3 | 5.3 | 5.3 | 5.3 |
| | C. albicans | 3.5 | 3.2 | 3.8 | 3.1 |
| | A. niger | 2.3 | 1.4 | 1.2 | 1.9 |
| 28 days | E. coli | 5.3 | 5.3 | 5.3 | 5.3 |
| | S. aureus | 5.1 | 5.1 | 5.1 | 5.1 |
| | Ps. aerug. | 5.3 | 5.3 | 5.3 | 5.3 |
| | C. albicans | 5.1 | 5.1 | 5.1 | 5.1 |
| | A. niger | 2.4 | 1.9 | 2.2 | 2.0 |

With respect to E. coli, high-viscosity HPMC appears to have a negative effect on PET. Results for other species were mixed. In no time period and in no species, however, did the presence or absence of PEG or glycerin appear to affect PET.

As noted above, the presence of zinc ions has a significant, positive effect on PET. In order to assess the impact of other ions on PET, the effect of PET was measured for four compositions, each lacking either potassium, magnesium, calcium, or all three ions, as compared to a composition containing all three ions. The formulation of each composition is shown in Table 16. The effect of each composition on PET is shown in Table 17.

TABLE 16
Compositions Having Varying Ions

| | Amount (% w/w) | | | | |
|---|---|---|---|---|---|
| | ophthalmic | ophthalmic | ophthalmic | ophthalmic | ophthalmic |
| | base with all | base without | base without | base without | base without |
| Ingredients | the ions | Potassium | Magnesium | Calcium | K, Mg, Ca |
| boric acid | 0.82 | 0.82 | 0.82 | 0.82 | 0.82 |
| sodium borate | 0.18 | 0.18 | 0.18 | 0.18 | 0.18 |
| zinc chloride | 0.0025 | 0.0025 | 0.0025 | 0.0025 | 0.0025 |
| ascorbic acid | 0.05 | 0.05 | 0.05 | 0.05 | 0.05 |
| glycerin | 0.25 | 0.25 | 0.25 | 0.25 | 0.25 |
| PEG 400 | 1.15 | 1.15 | 1.15 | 1.15 | 1.15 |
| sodium | 0.0005 | 0.0005 | 0.0005 | 0.0005 | 0.0005 |
| phosphate | | | | | |
| potassium | 0.01 | — | 0.01 | 0.01 | — |
| chloride | | | | | |
| magnesium | 0.01 | 0.01 | — | 0.01 | — |
| chloride | | | | | |
| calcium | 0.01 | 0.01 | 0.01 | — | — |
| chloride | | | | | |
| dextrose | 0.005 | 0.005 | 0.005 | 0.005 | 0.005 |
| Sodium lactate | 0.05 | 0.05 | 0.05 | 0.05 | 0.05 |
| 60% solution | | | | | |
| glycine | 0.00002 | 0.00002 | 0.00002 | 0.00002 | 0.00002 |
| purified water | q.s. to 100 | q.s. to 100 | q.s. to 100 | q.s. to 100 | q.s. to 100 |

TABLE 17
Effect of Ions on PET

| | | Log Reduction | | | | |
|---|---|---|---|---|---|---|
| No. of | | Ophthalmic | Ophthalmic | Ophthalmic | Ophthalmic | Ophthalmic |
| Days | | Base with all | Base without | Base without | Base without | Base without |
| incubated | Organism | the ions | Potassium | Magnesium | Calcium | K, Mg, Ca |
| 24 hrs | E. coli | 0.7 | 0.2 | 1.0 | 0.4 | 1.2 |
| | S. aureus | 0.0 | 0.1 | 0.0 | 0.2 | 0.4 |
| | Ps. aerug. | 0.8 | 0.8 | 0.8 | 0.5 | 0.7 |
| | C. albicans | 0.6 | 0.7 | 0.7 | 0.5 | 0.5 |
| | A. niger | 1.9 | 1.3 | 2.0 | 2.0 | 1.5 |
| 3 days | E. coli | 0.2 | 0.2 | 0.9 | 0.7 | 1.4 |
| | S. aureus | 0.6 | 0.0 | 0.1 | 0.5 | 0.9 |
| | Ps. aerug. | 1.0 | 1.2 | 1.1 | 0.2 | 0.5 |
| | C. albicans | 0.6 | 0.6 | 0.2 | 0.1 | 0.5 |
| | A. niger | 2.0 | 2.7 | 2.2 | 2.0 | 2.3 |
| 7 days | E. coli | 0.2 | 0.0 | 1.1 | 0.8 | 1.9 |
| | S. aureus | 1.6 | 1.5 | 1.2 | 1.9 | 2.2 |
| | Ps. aerug. | 0.7 | 0.7 | 0.8 | 0.6 | 0.7 |
| | C. albicans | 1.4 | 1.5 | 1.7 | 1.1 | 1.2 |
| | A. niger | 1.8 | 1.7 | 2.3 | 1.9 | 1.3 |
| 14 days | E. coli | 0.2 | 0.0 | 1.6 | 1.1 | 2.1 |
| | S. aureus | 3.3 | 3.1 | 3.5 | 3.6 | 4. |
| | Ps. aerug. | 0.6 | 0.6 | 0.3 | 0.5 | 1.5 |
| | C. albicans | 2.2 | 2.5 | 2.8 | 2.1 | 2.8 |
| | A. niger | 1.7 | 2.7 | 2.0 | 2.1 | 2.2 |
| 21 days | E. coli | 0.0 | −0.1 | 1.2 | 1.0 | 1.8 |
| | S. aureus | 4.2 | 4.5 | 4.9 | 4.9 | 4.9 |
| | Ps. aerug. | 0.3 | −0.8 | −0.4 | 0.4 | 1.3 |
| | C. albicans | 3.1 | 2.7 | 3.6 | 2.7 | 4.0 |
| | A. niger | 1.9 | 1.9 | 1.8 | 2.3 | 1.7 |
| 28 days | E. coli | 0.1 | 0.3 | 1.2 | 1.0 | 1.2 |
| | S. aureus | 4.9 | 4.9 | 4.9 | 4.9 | 4.9 |
| | Ps. aerug. | 0.3 | 0.3 | 0.2 | 0.1 | 1.2 |
| | C. albicans | 3.1 | 3.2 | 3.9 | 3.1 | 4.3 |
| | A. niger | 1.9 | 2.1 | 2.0 | 1.9 | 2.6 |

As can be seen in Table 17, with respect to E. coli, the removal of magnesium ions or potassium, magnesium, and calcium ions both result in consistent improvement in PET during all time periods. Results for other compositions were mixed, although by day 28, PET was improved in A. niger using any ion-lacking composition and by day 14 in C. albicans using compositions lacking either magnesium or potassium, magnesium, and calcium.

Thus, an illustrative embodiment of a composition of the present invention comprises an antimicrobial buffer, such as a borate buffer, and an antimicrobial metal ion, such as a zinc ion. Other ingredients may also be included, such as a demulcent, a surfactant, ascorbic acid, and/or a chelating agent.

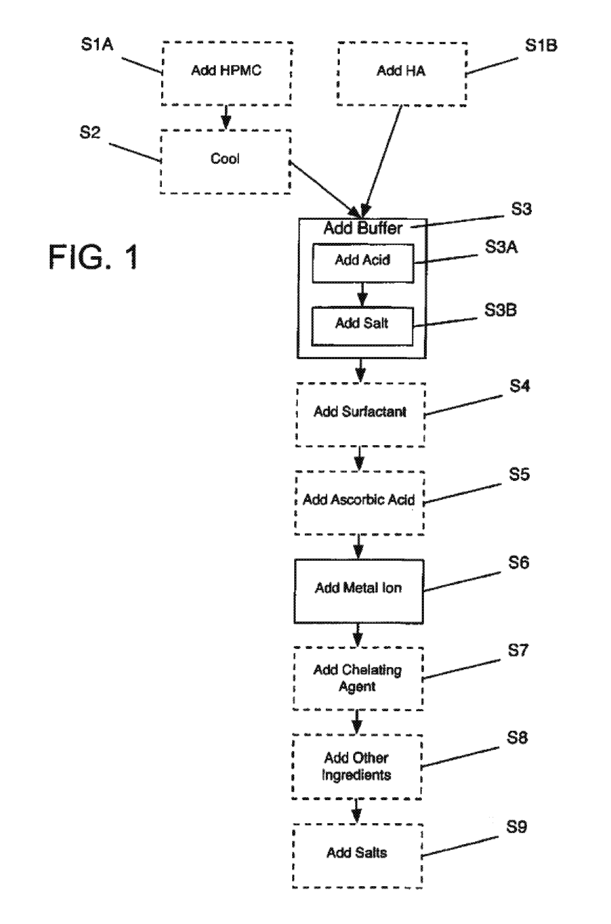

A flow diagram of an illustrative method for preparing such a composition according to the invention is shown in FIG. 1. At optional step S1A, in the case that the composition is to comprise HPMC, the HPMC is dispersed under vigorous stirring in a quantity of water at 70° C. to 90° C. equal to approximately half the total volume of the composition, followed by cooling at optional step S2. Alternatively, in the case that the composition is to comprise HA, at optional step S1B, the HA is dissolved in a quantity of room temperature water equal to approximately half the total volume of the composition.

For all compositions not comprising HPMC or HA, preparation may begin at step S3, wherein the buffer system is added to a quantity of water equal to approximately half the total volume of the composition. Step S3 includes the addition of an acid (e.g., boric acid) at step S3A and a salt (e.g., sodium borate) at step S3B. Steps S3A and S3B may be performed in the order opposite that shown in FIG. 1.

Next, in the case that the composition will include a surfactant or ascorbic acid, these are added at optional steps S4 and S5, respectively. At step S6, a source of metal ions is added. As noted above, while compositions according to the invention have been described as including a zinc ion, other metal ions exhibiting antimicrobial properties may also be used.

A chelating agent, if desired, may be added at optional step S7. Chelating agents useful in the present invention include, but is not limited to, amino carboxylic acid compounds or water-soluble salts thereof, including ethylenediaminetetraacetic acid, nitrilotriacetic acid, diethylenetriamine pentaacetic acid, hydroxyethylethylenediaminetriacetic acid, 1,2-diaminocyclohexanetetraacetic acid, ethylene glycol bis (beta-aminoethyl ether) in N,N,N′,N′tetraacetic acid (EGTA), aminodiacetic acid and hydroxyethylamino diacetic acid. These acids can be used in the form of their water soluble salts, particularly their alkali metal salts. Certain embodiments of the present invention incorporate the di-, tn- and tetra-sodium salts of ethylenediaminetetraacetic acid (EDTA).

Other chelating agents such as citrates and polyphosphates can also be used in the present invention. The citrates which can be used in the present invention include citric acid and its mono-, di-, and tri-alkaline metal salts. The polyphosphates which can be used include pyrophosphates, triphosphates, tetraphosphates, trimetaphosphates, tetrametaphosphates, as well as more highly condensed phosphates in the form of the neutral or acidic alkali metal salts such as the sodium and potassium salts as well as the ammonium salt. Amino acids such as glutamic and aspartic acids can also be used. Mixtures of the above chelating agents may be incorporated herein.

The chelating agents may be employed at about 0.0001 to about 1.0 weight percent of the composition, optionally at about 0.001 to about 0.5 weight percent, or optionally about 0.01 to about 0.3 weight percent.

At optional step S8, other ingredients may be added, such as medicaments or other therapeutic agents. Salts, if necessary or desired, may be added at optional step S9. As will be recognized by one skilled in the art, the composition may then be brought to a desired volume or weight by adding water and then optionally be mixed and/or filtered. Optionally, the final composition has undergone sterilization by filtering.

If the concentration of zinc chloride or zinc sulfate is higher than 0.01%, it starts to precipitate around pH 7.4. That is, the tendency of zinc to precipitate increases as pH rises above about 7.4. The addition of ascorbate (e.g., ascorbic acid) keeps zinc in solution. The addition of other salts, such as sodium chloride, also helps the solubility of zinc, particularly where zinc is present in concentrations greater than 0.01%, although a large quantity of sodium chloride is needed. Other ingredients can be used to form highly-soluble salts with zinc, thereby improving zinc's solubility. Such ingredients include, for example, oxalic acid, sodium fluoride, sodium nitrate, lactic acid, and sodium iodide.

Optionally, certain embodiments incorporate ascorbic acid and a zinc ion source for two reasons. First, a comparatively small quantity of ascorbic acid is needed to achieve an improvement in zinc solubility. Second, ascorbic acid may also be used to resolve an incompatibility between zinc chloride and polysorbate 80. Solubilizers, such as polysorbate 80, are often used if a composition is to contain a lipophilic compound, such as latanoprost, menthol, and benzophenone. Table 18 shows formulations for a vehicle and latanoprost-containing composition according to the invention, each containing zinc chloride and polysorbate 80. Table 19 shows similar formulations for a vehicle and composition further comprising timolol maleate.

Surfactants can perform multiple functions in these types of formulations, besides dissolving lipophilic materials such as latanoprost. Certain of the embodiments of the present invention incorporate nonionic surfactants.

The surface active agents having antimicrobial activity may be employed at about 0.001 to about 5 weight percent of the composition, optionally at about 0.005 to about 3 weight percent, or optionally about 0.01 to about 1.2 weight percent.

When used herein, the nonionic surfactant Polysorbate 80 can increase the preservative efficacy of the formulations, as shown above in Table 5. Further, polysorbate 80 is a known penetration enhancer, so it can help in pushing the drugs through a user's cornea. Finally, polysorbate 80 is an accepted demulcent. So it would also help in reducing the irritation, if there is any, due to the API or due to some other reason. Hence, it is generally desirable to include polysorbate 80 in formulations according to the invention.

Similarly, zinc is very beneficial for improving preservative efficacy. Ascorbic acid is useful in keeping these two beneficial but mutually incompatible ingredients (polysorbate 80 and zinc) in solution. Ascorbic acid also contributes to the preservative efficacy. Unfortunately, ascorbic acid is unstable in solution, so one should not rely exclusively on the preservative efficacy of ascorbic acid during the entirety of the shelf life of the product. Hence, for formulations containing ascorbic acid, the preservative efficacy was determined after storing the product at 40° C. for a period of time in order to degrade ascorbic acid. Interestingly, ascorbic acid overcame the incompatibility between zinc and polysorbate 80 even after its own degradation.

TABLE 18
Ascorbic Acid-Stabilized Compositions Containing Zinc

Amount (% w/w)

| | | Composition with |
|---|---|---|
| Ingredients | Vehicle | latanoprost |
| Latanoprost | — | 0.005 |
| Boric Acid | 0.8 | 0.8 |
| Sodium Borate | 0.2 | 0.2 |
| Ascorbic Acid | 0.01 to 0.25 | 0.01 to 0.25 |
| Zinc chloride | 0.01 | 0.01 |
| Polysorbate 80 | 1.0 | 1.0 |
| Sodium Chloride | 0.1 to 0.25 | 0.1 to 0.25 |
| Purified Water | q.s. to 100 | q.s. to 100 |

TABLE 19
Ascorbic Acid-Stabilized Compositions Containing Zinc

Amount (% w/w)

| | | Formulation with |
|---|---|---|
| Ingredients | Vehicle | latanoprost |
| Latanoprost | — | 0.005 |
| Timolol Maleate | — | 0.5-1.0 |
| Boric Acid | 0.8 | 0.8 |
| Sodium Borate | 0.2 | 0.2 |
| Ascorbic Acid | 0.01 to 0.25 | 0.01 to 0.25 |
| Zinc chloride | 0.01 | 0.01 |
| Polysorbate 80 | 1.0 | 1.0 |
| Sodium Chloride | 0.1 to 0.25 | 0.1 to 0.25 |
| Purified Water | q.s. to 100 | q.s. to 100 |

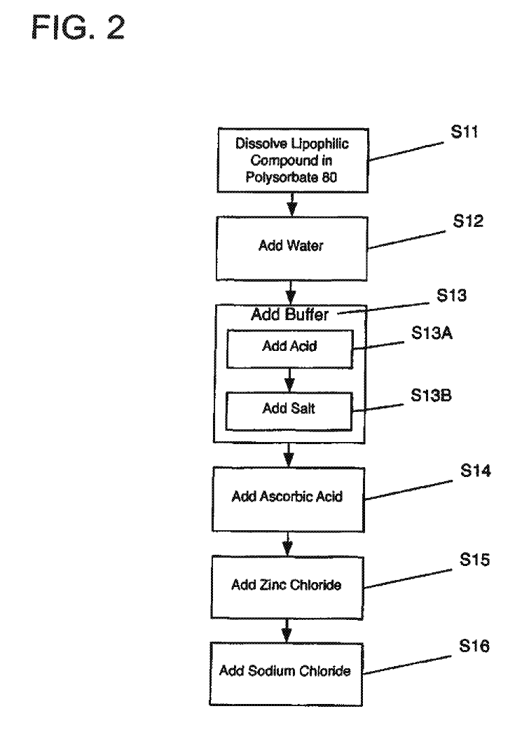

A flow diagram of an illustrative method for preparing a composition such as those of Tables 18 and 19 is shown in FIG. 2. At step S11, the lipophilic compound (latanoprost, in the examples in Tables 18 and 19) is dissolved in polysorbate 80. At step S12, a quantity of water equal to approximately half the total volume of the composition is added to the lipophilic compound and polysorbate 80 of step S11. Next, at step S13, the buffer system is added by the addition of an acid (boric acid, in the examples in Tables 18 and 19) at step S13A and a salt (sodium borate, in the examples in Tables 18 and 19) at step S13B. Ascorbic acid, zinc chloride, and sodium chloride are then added at steps S14, S15, and S16, respectively. Optionally, the osmolality of the composition is adjusted by manipulation of the ratio of sodium chloride and ascorbic acid to a value between about 200 mOsm/kg and about 400 mOsm/kg, optionally between about 250 mOsm/kg and about 330 mOsm/kg, and optionally to about 290 mOsm/kg.

An additional benefit of an ascorbic acid-stabilized composition such as those above is that it avoids the typical yellow discoloration caused by the degradation of ascorbic acid. In the presence of zinc, such discoloration does not develop. As such, discoloration of any solution containing ascorbic acid, not just the ophthalmic solutions described above, may be avoided by the addition of a quantity of zinc.

In addition to the preservative effects of metal ions described above, it is known that zinc, in particular, exhibits an astringent effect, i.e., is capable of precipitating some proteins from solution. It is also know that some proteins found on the surface of the eye, whether by direct secretion or transport there, exhibit an allergenic effect resulting in excessive watering, redness, itching, and other symptoms typical of ocular allergic reactions. For example, macrophage inflammatory protein-1α (MIP-1α) has been identified as involved in hypersensitivity reactions in the conjunctiva. See Miyazaki et al., Macrophage inflammatory protein-1α as a co-stimulatory signal for mast cell-mediated immediate hypersensitivity reactions, J. Clin. Invest., 115(2):434-442 (2005), which is hereby incorporated by reference. Without being limited by theory, it is believed that proteins are associated with similar allergenic reactions in the ear and nasal passages, such as otitis media and rhinitis, respectively.

Compositions of the present invention include, therefore, compositions containing an effective amount of zinc, which may be useful in precipitating one or more proteins from a surface of the eye, ear, or nose, thereby relieving or reducing an allergy symptom caused by the protein. As used herein, “an effective amount” shall include amounts capable of precipitating from a surface of a user's eye, ear, or nasal passage, at least one protein causing a symptom of an ocular, otic, or nasal allergic reaction. In addition, such a composition may further comprise an antiallergy compound in order to further reduce allergy symptoms. Suitable antiallergy compounds include, for example cetirizine, olopatadine, cromolyn sodium, nephazoline, pheniramine, levocabastine, pemirolast, oxymetazoline, loratadine, tetrahydrozoline, nedocromil, and azelastine.

The foregoing description of various aspects of the invention has been presented for purposes of illustration and description. It is not intended to be exhaustive or to limit the invention to the precise form disclosed, and obviously, many modifications and variations are possible. Such modifications and variations that may be apparent to a person skilled in the art are intended to be included within the scope of the invention as defined by the accompanying claims.

1-29. (canceled)

30. A method for treating an allergy symptom in an individual, the method comprising:
administering to a surface of at least one of an eye, an ear, and a nasal passage of an individual a composition comprising an effective amount of zinc,
wherein the zinc is capable of precipitating from the administered surface at least one protein causing a symptom of an allergic reaction.

31. The method of claim 30, wherein the effective amount of zinc includes at least one soluble salt of zinc selected from a group consisting of: zinc chloride, zinc sulfate, zinc acetate, and zinc lactate.

32. The method of claim 30, wherein the composition further comprises a quantity of ascorbic acid capable of inhibiting precipitation of zinc from the composition.

33. The method of claim 30, wherein the composition further includes an antimicrobial buffer.

34. The method of claim 30, wherein the composition further includes at least one antiallergy compound.

35. The method of claim 34, wherein the at least one antiallergy compound is selected from a group consisting of: cetirizine, olopatadine, cromolyn sodium, nephazoline, pheniramine, levocabastine, pemirolast, oxymetazoline, loratadine, tetrahydrozoline, nedocromil, and azelastine.

36. A composition comprising:
a source of zinc,
wherein the source of zinc is capable of precipitating from a surface of at least one of an eye, an ear, and a nasal passage, at least one protein capable of causing at least one symptom of an allergic reaction.

37. The composition of claim 36, wherein the source of zinc includes at least one soluble salt of zinc selected from a group consisting of: zinc chloride, zinc sulfate, zinc acetate, and zinc lactate.

38. The composition of claim 36, further comprising: a quantity of ascorbic acid capable of inhibiting precipitation of zinc from the composition.

39. The composition of claim 36, further comprising an antimicrobial buffer.

40. The composition of claim 36, further comprising at least one antiallergy compound.

41. The composition of claim 40, wherein the at least one antiallergy compound is selected from a group consisting of: cetirizine, olopatadine, cromolyn sodium, nephazoline, pheniramine, levocabastine, pemirolast, oxymetazoline, loratadine, tetrahydrozoline, nedocromil, and azelastine.
